(19) United States
(12) Patent Application Publication
(10) Pub. No.: US 2004/0018614 A1
(43) Pub. Date: Jan. 29, 2004

(54) Biochip preparation method

(75) Inventors: Yu-Ting Lin, Taipei (TW); Yu-Chang Shen, Taipei (TW); In-Shan Sir, Kaohsiung (TW)

Correspondence Address: Ladas & Parry, Suite 2100, 5670 Wilshire Boulevard, Los Angeles, CA, 90036, US

(73) Assignee: BENQ CORPORATION

(21) Appl. No.: 10/616,520

(22) Filed: Jul. 9, 2003

(30) Foreign Application Priority Data
Jul. 12, 2002 (TW) 91115598

(51) Int. Cl. C12M 1/34; B05D 3/00

(52) U.S. Cl. 435/287.2; 427/2.11

(57) ABSTRACT

A preparation method for biochips. The method comprises spraying a hydrophobic material onto a substrate by a micro injecting process to form a hydrophobic region thereon, wherein the hydrophobic region separates a plurality of partitions on the substrate, and immobilizing a probe on the partition by the micro injecting process.

### BACKGROUND OF THE INVENTION

1. Field of the Invention

The present invention relates to a preparation method for biochips. More particularly, the present invention relates to a preparation method for biochips using a micro-injecting process.

2. Description of the Related Arts

The Biochip technology has contributed greatly to life sciences since the end of the 20th century. In general, biochips are products for biochemical analysis. The analysis target can be a gene, a protein, or a tissue cell. The advantages of biochips include high reliability, high accuracy and speed. Small amounts of sample and reagent are needed. Furthermore, large-scale analysis can be made, and integrative and parallel data can be obtained.

Biochips include microarrays, DNA chips, protein/antibody chips, tissue chips, and labs-on-chip, etc. Only microarrays and DNA chips are mature products. The applications of biochips include gene sequencing, toxological analysis, pathogenic gene expression, single nucleotide polymorphisms (SNPs), medical jurisprudence, pharmaceutical screening, and the detection of biochemical weapons. The extensive applications for biochips make them popular in many fields.

Many studies of biochips focus on the preparation of high density biochips with low cost. Based on different probe preparations, biochips can be classified into three groups. The first one is manufactured by a light-directed synthesis which combines photolithography and chemical synthesis, and was developed by Affymetrix company. The second one is formed by a spotting method developed by Stanford University, in which pre-synthesized DNA, RNA, or protein is immobilized on a substrate by a manipulator. The third type uses a micro injecting process, as developed by Rosetta Inpharmatics, in which the complementary nucleotides (probes) are produced by the micro injecting process. A sample containing targets labeled by fluorescent, or enzyme labeling is hybridized with the chip and the data is read and analyzed by computer programs.

Current commonly used preparations for the biochips include a pin method and a micro injecting process. The micro injecting process further includes a piezo type, for example, U.S. Pat. Nos. 5,985,551 and 6,177,558B1 proposed by Protogene, and a thermal bubble type. The drawback of piezo type micro injecting process is its low density of about 10-10⁴ spots/cm².

Quality, resolution and cost are critical factors for the preparation of biochips. The above mentioned methods, such as photolithography, pin method, or piezo type micro injecting process, have several drawbacks in practice. Micro injecting processes also have contamination problems between probe spots. To overcome the contamination problems, U.S. Pat. No. 5,552,270 discloses a matrix consisting of a multiplicity of portions. The portions are separated from one another by interstices of polyacrylamide and have a thickness not exceeding 30 μm. These portions prevent contamination between probe spots and increase the number of immobilized probes; however the preparation is complicated and costly. Furthermore, the biochips must be stored in non-volatile oil since the water of the probes solution may vaporize. This storage results in an additional step of chloroform or ethanol washing. Therefore, this type of biochip is not convenient in practice.

### SUMMARY OF THE INVENTION

It is therefore an object of the present invention to provide a simple and economic preparation method for biochips. The preparation method comprises: applying a micro-injecting process to spray a hydrophobic material on a substrate for forming a hydrophobic region thereon, and a plurality of partitions being defined on the hydrophobic region, and immobilizing a probe on each partitions by the micro-injecting process. The preparation of the present invention prevents interference between probes and enhances the density of the probe as well as the resolution of the biochip. With the preparation of the present invention, biochips with high density, small spots, high resolution, and low cost can be obtained.

### BRIEF DESCRIPTION OF THE DRAWINGS

The present invention will be more fully understood and further advantages will become apparent when reference is made to the following description of the invention and the accompanying drawings in which:

FIGS. 1A-1B are diagrams showing spraying of hydrophobic materials on a substrate by a micro injecting process of the present invention. FIG. 1A shows vertical spraying and FIG. 1B horizontal spraying.

FIGS. 2A-2B are diagrams showing preferred embodiments of the partitions on the substrate in the present invention. FIG. 2A show square partitions, and FIG. 2B show circular partitions.

FIG. 3 is a cross-section showing the hydrophobic material 18 disposed on the substrate 16 and the partitions 21 covered with the probe spots 22.

FIGS. 4A-4D are diagrams showing immobilization of a nucleic acid probe 24 onto the partitions 20 of the substrate 16. FIG. 4A shows a micro injecting process of a nucleic acid 24 with protecting group 26 onto different, hydrophilic partitions 20; FIG. 4B shows de-protection by an acidic solution 28; FIG. 4C shows a micro injecting process of a second layer of nucleic acid 24 with protecting group 26 onto the partitions 20; and FIG. 4D shows a completed biochip.

FIG. 5 is a diagram showing the thermal bubble micro-injector used in the present invention.

FIGS. 6A-6D are diagrams showing the micro-injecting process of the present invention.

### DETAILED DESCRIPTION OF THE INVENTION

Without intending to limit it in any manner, the present invention will be further illustrated by the following description.

The preparation method of biochips as shown in the present invention comprises steps of applying a micro-injecting process to spray a hydrophobic material on a substrate for forming a hydrophobic region thereon, defining a plurality of partitions on the hydrophobic region, and immobilizing a probe on each partitions by the micro-injecting process. The preparation method prevents interference between probes and enhances the density of the probe as well as the resolution of the biochip.

The substrate of the present invention can be a hydrophobic or a hydrophilic substrate. The hydrophobic substrate can include, but is not limited to, glass, silicon, plastic, nylon, resin, quartz, mica, ceramics, or metals. The hydrophilic substrate can be made by, but is not limited to, polystyrene, polyester, polycarbonate, polyvinylchloride, polyethylene, polypropylene, polysulfone, polyurethane, or polymethylmethacrylate (PMMA).

When a hydrophobic substrate is applied, after the partitions are formed, a hydrophilic treatment to the partitions is needed to add hydrophilic groups on the surface of the partitions before the binding process of probes. The hydrophilic group can be —NH₂, —COOH, —SH, epoxide, aldehyde, or streptavidin.

When the hydrophilic substrate is used, the surface of the substrate is modified to be hydrophobic before the partitions are formed. In addition, a hydrophilic treatment on the partition is needed after the partitions are formed. Therefore, hydrophilic groups are added to the surface of the partition before binding with probes. Details for the surface modification of the substrate are disclosed in several articles and will not be described further in this application.

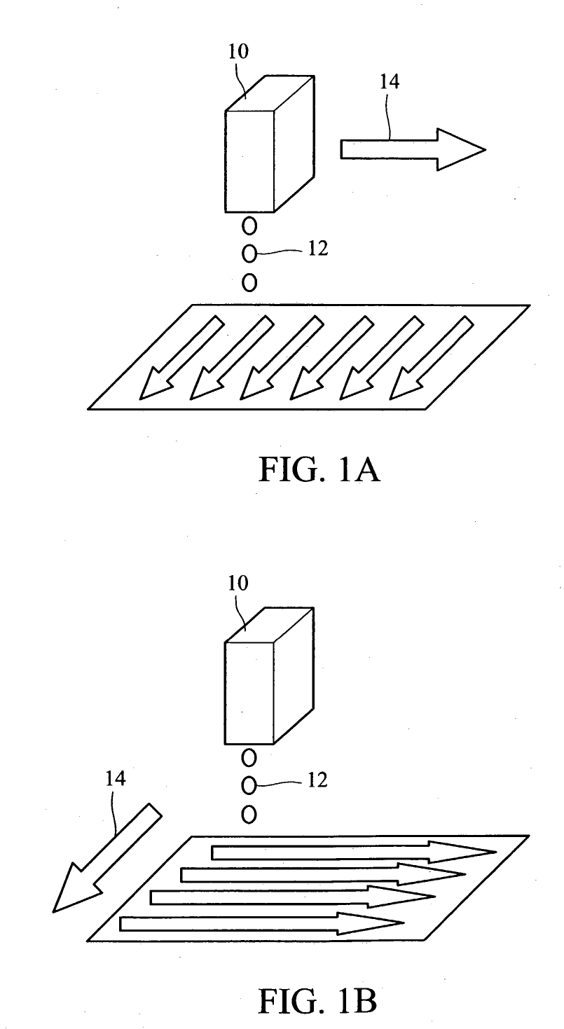

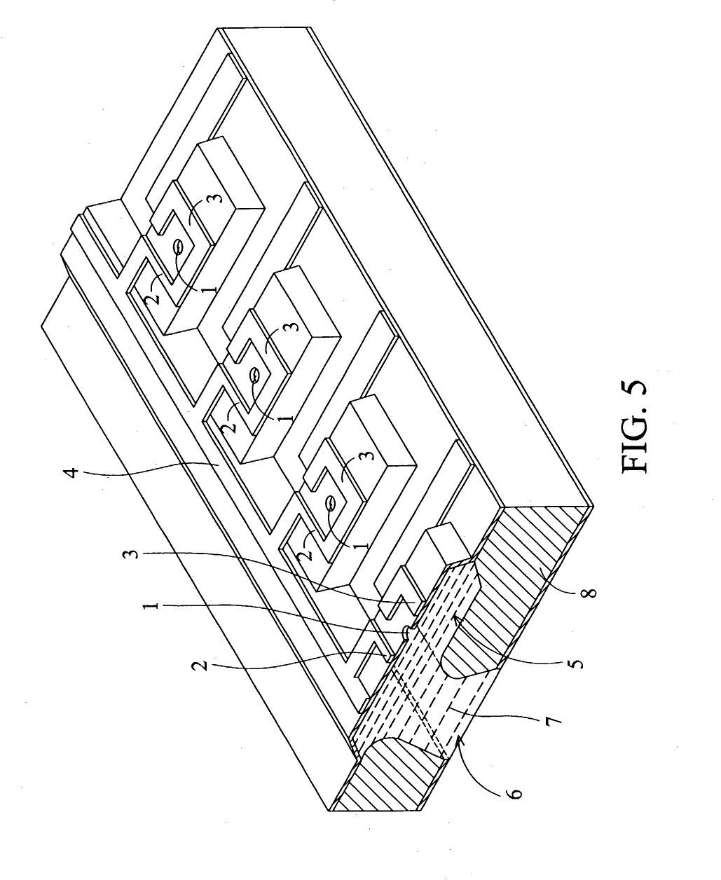

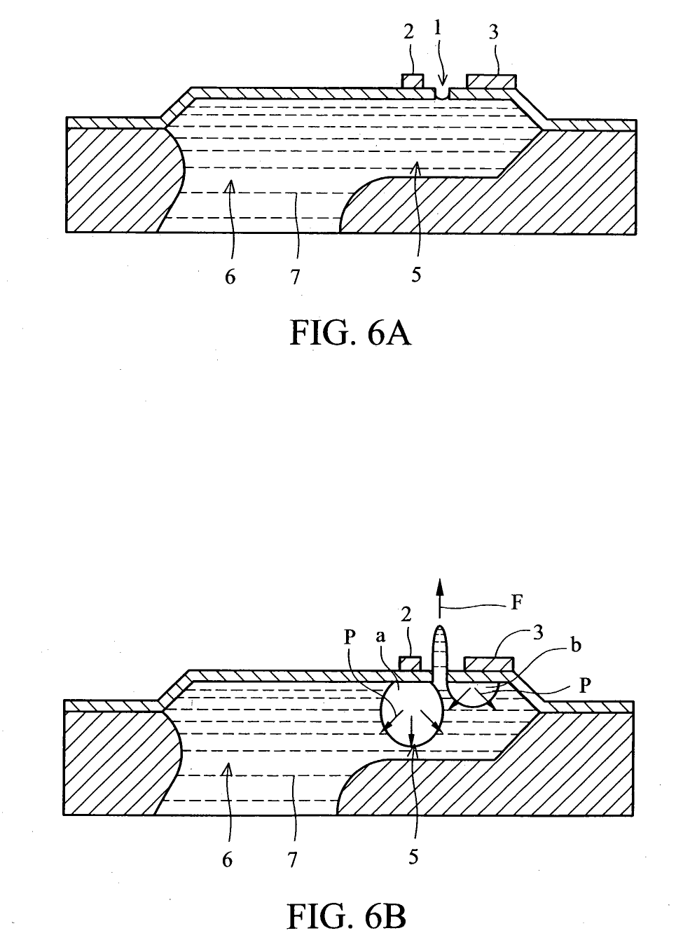

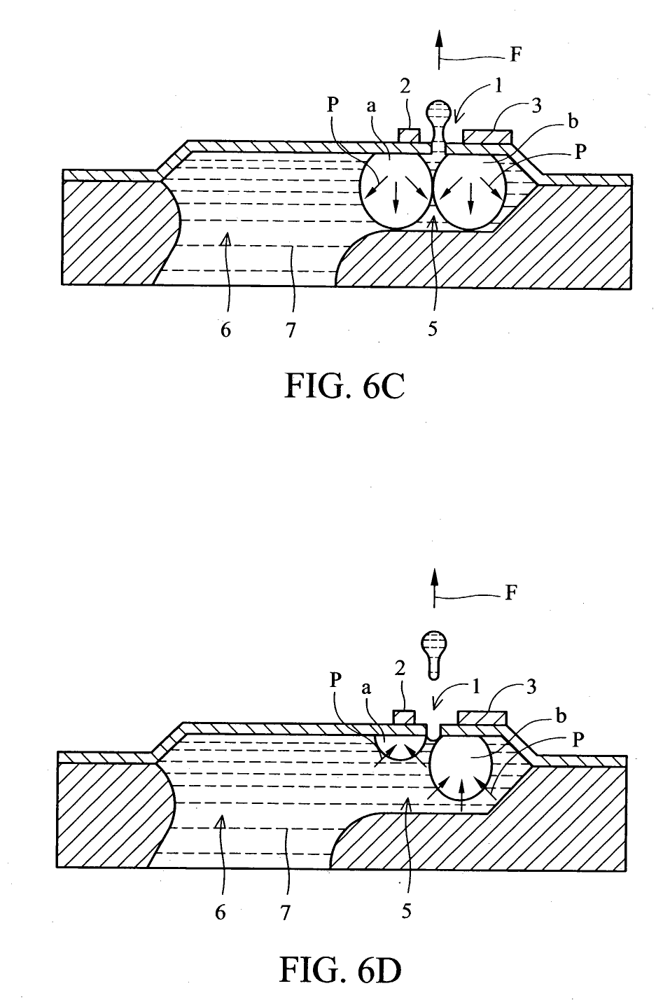

As shown in FIGS. 1A and 1B of the present invention, the micro-injector 10 sprays drops 12 vertically, horizontally, unidirectionally or bidirectionally. The micro-injector 10 moves in a direction 14. The micro-injector includes thermal bubble or piezo electric micro-injectors. As shown in FIGS. 5 and 6A-6D, the thermal bubble micro-injector includes a chamber 5 for a fluid, a micro injecting process pore 1 disposed on the chamber 5 for ejecting fluid, and a first heater 2 and a second heater 3 arranged on the two sides of the pore 1 respectively. An electrode 4 is arranged on the micro-injector 10 for power supply. When the chamber 5 is occupied with fluid 7, the first heater 2 produces a first bubble a, and the second heater 3 then produces a second bubble b. The two bubbles disconnect fluid 7 and then spray out a drop of fluid F. In addition, the first and second heaters 2, 3 are triggered by only one signal, and the production of the first bubble acts as a valve to limit the fluid flow.

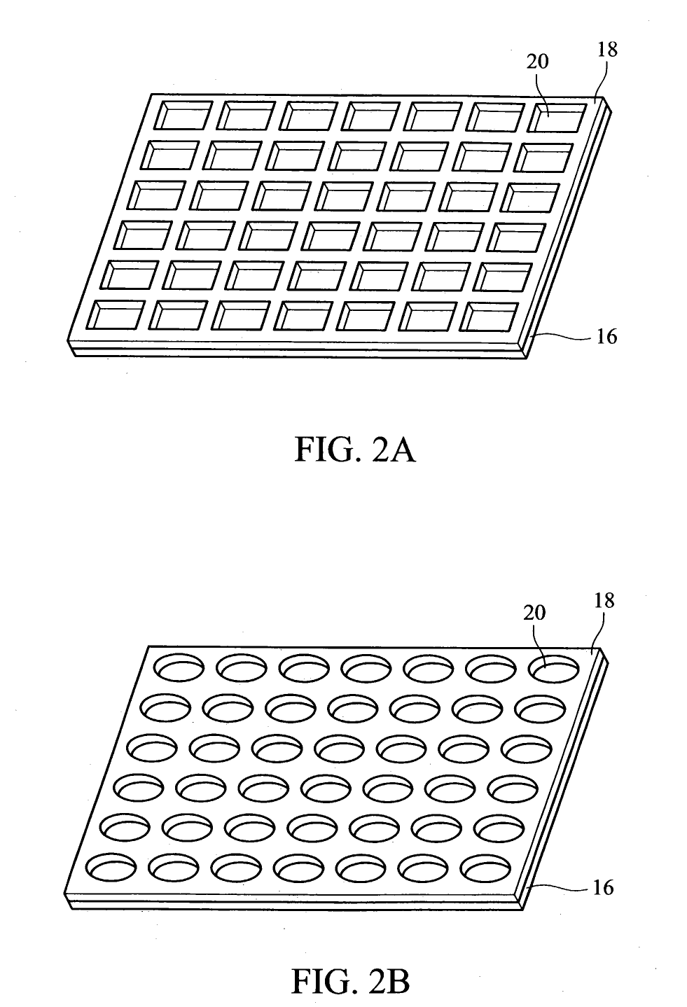

The hydrophobic material sprayed by the micro injecting process can include, but is not limited to, Teflon, polyimide, fluoro-compound, or silicon compound. The hydrophobic material forms a hydrophobic region 18 on the substrate 16 and are also defined a plurality of partitions 20. The partitions 20 can be square, circular, or any other geometric figures, as shown in FIGS. 2A and 2B. Width of each partition 20 is within 20˜200 μm², and the hydrophobic region 18 has a thickness of about 1˜30 μm and a width of 5˜100 μm².

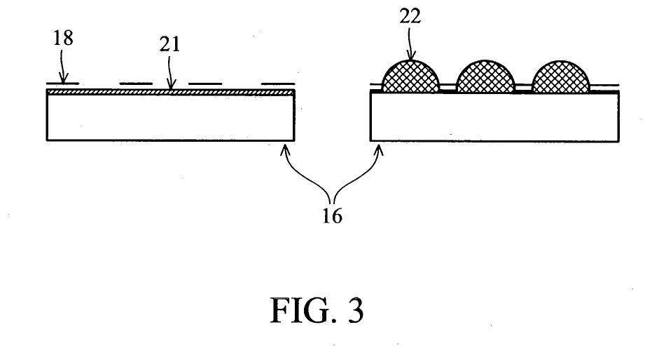

As shown in FIG. 3, the probe 22 is sprayed in a drop form by the micro-injector. The sprayed probes can cover the hydrophilic partition 21 on the substrate 15. The hydrophobic region 18 is not covered by the probe 22. The micro-injector contains the probe solution, which can be DNA, RNA, nucleotides, oligonucleotides, or protein. The probe is immobilized on the substrate via a functional group. The binding of the functional group can include, but is not limited to, adsorption, covalent binding, encapsulation, cross-linking, or entrapment. The probe solution of DNA, RNA, or nucleotides can be modified to be phosphonate, hydrogen phosphonate, phosphonamidite, phosphoamidite, phosphate, phosphoramidite, phosphite, or methylphosphonamidite to enhance binding with functional groups of the substrate. In oligonucleotide microarray, the 5′ or 3′ ends of the nucleotides are protected by a protecting group to prevent binding between nucleotides in the same layer.

The determinant factors for small drop in a microarray of a biochip include surface tension or viscosity of the nucleotide solution, and size of the micro injecting process pore. Each drop is usually 25˜250 μm.

Using a thermal micro-injector is used to prepare a nucleotide microarray of the biochip, the solvent property such as viscosity must be taken into consideration. In general, the smaller the drop size, the more volatile the drop. Therefore, the solution usually contains at least one high b.p. solvent, for example, a solvent with a b.p. higher than 140° C., to prevent drop vaporization. Suitable solvents are polar and proton-free, such as dinitriles, mononitriles, glymes, diglymes, triglymes, trimethylphosphates, dimethylformamides (DMF), or N-methylpyrrolidinone (NMP).

In the preparation of an oligonucleotide microarray of the biochip, the protected nucleotides are sequentially immobilized on the substrate by the micro-injector. The protecting group of the nucleotide prevents overlapping of nucleotides in the same layer. After the first layer of nucleotides is immobilized, the protecting group of the nucleotides can be removed by an acidic solution. After that, the second layer of nucleotides can formed to bind to the first layer of the nucleotides. Sequentially, the desired biochip can be obtained. This method is more flexible in designing probes. In the preparation of DNA, RNA, or protein biochips, the pre-synthesized DNA, RNA, or protein is sprayed onto the partition of the substrate. In the preparation of peptide biochips, the process is similar to nucleotide microarray, and the peptides are linked one by one.

The biochip is then hybridized with sample to be analyzed. After the hybridization, the labels the analyzed sample are screened and analyzed in a computer. The labels may be fluorescent labels, radio-labels, or enzyme-linked labels.

Practical examples are described herein.

### EXAMPLE 1

### First Preparation of Batch-Type Oligonucleotide Microarray by a Thermal Micro-Injector

1. Partitions Formed on Glass by Micro-Injector

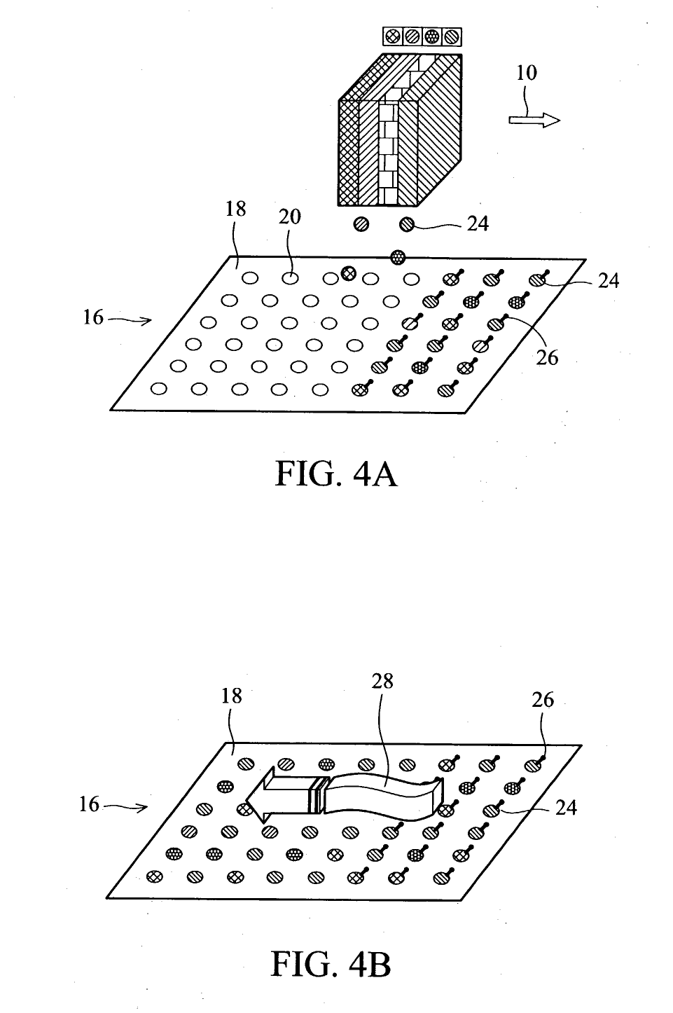

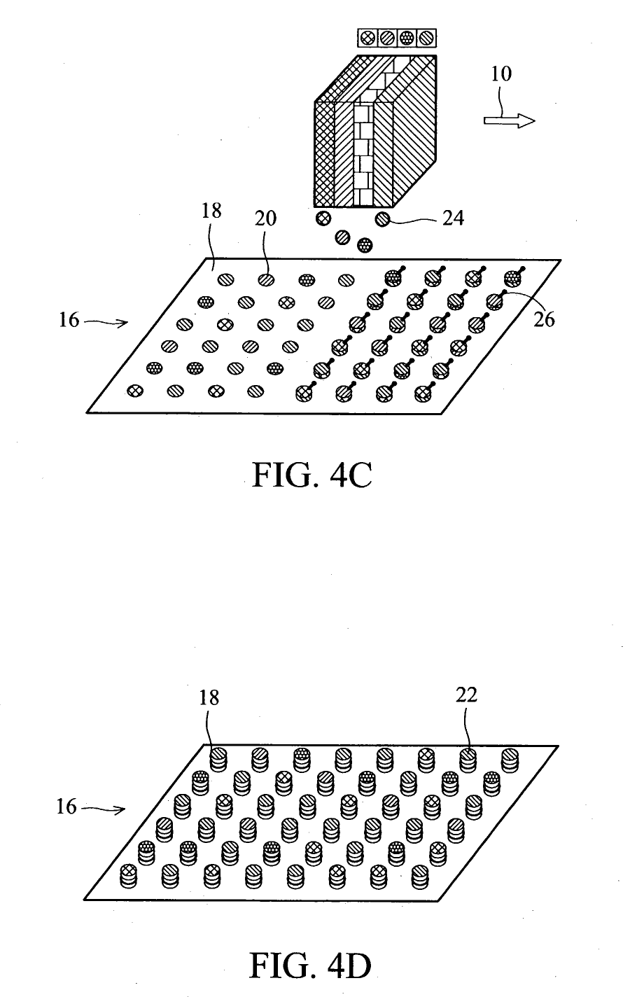

Please refer to FIGS. 4A to 4D. A hydrophobic material, such as Teflon, is sprayed onto the surface of a substrate 16, such as glass, by a thermal micro-injector to form a hydrophobic region 18 which separates a plurality of square partitions 20 as shown in FIG. 2A. The width of the partition is about 50 μm. The hydrophobic region 18 has a thickness of about 2 μm and a width of about 5 μm. A microarray is then formed, and the surface of the partitions is hrdrophobic.

2. Hydrophilic Treatment of the Partitions

The square partitions are then silanized by micro-injecting octyltrichlorosilane. The surface of the partitions becomes hydrophilic because of —SH groups which enable the partitions to bind nucleotide probes. Detailed steps are disclosed in U.S. Pat. No. 6,159,695.

3. Oligonucleotides Bound to the Microarray

DMTr-nucleotide phosphoramidite containing tetrazole is sprayed onto the silanized partitions 21 by a micro injecting process. A first layer of nucleotides 24 is bound to —SH group on the hydrophilic partitions 21. The deprotection is then performed by using an acidic solution 28 such as trichloroacetic acid or hydrogen chloride as shown in FIG. 4B. After that, a second layer of nucleotides 24 is sprayed onto the surface 16. Sequentially, nucleotides are connected to one another. Finally, an oligonucleotide microarray is obtained. Details steps can be found in U.S. Pat. No. 5,985,551 or U.S. Pat. No. 6,184,347B1.

4. Hybridization Performed on the Oligonucleotide microarray.

A labeled sample is hybridized with the oligonucleotide microarray, and then analyzed the hybridized compound from the label. After that, the exact sequence of the sample can be identified. Details can be found in U.S. Pat. No. 5,985,551.

### EXAMPLE 2

### Second Preparation of Batch-Type Oligonucleotide Microarray

The partitions of the oligonucleotide microarray can also be circular, as shown in FIG. 2B. The circular partitions 20 can be formed by directly spraying the hydrophobic material on the substrate 16 or by spraying the hydrophobic material on the substrate 16 covered with a plurality of circular masks (not shown). The circular masks are then removed, and the circular partitions are formed. The subsequent process is similar to Example 1.

### EXAMPLE 3

### Preparation of Protein Chip Using Piezo Micro-Injector

1. Partitions Formed on Glass by a Micro-Injector

A hydrophobic material, such as polyimide, is sprayed onto the surface of a substrate such as glass, by a piezo micro-injector to form a hydrophobic region which separates a plurality of square partitions on the substrate as shown in FIG. 2A. The width of the partition is about 50 μm. The hydrophobic material has a thickness of about 2 μm and a width of about 5 μm. A microarray is then formed.

2. Protein Bound to the Microarray

The square partitions are then silanized by micro-injecting octyltrichlorosilane. Protein is then bound to the surface of the substrate. Details can be found in U.S. Pat. No. 6,225,047B1.

3. Detection of Target Protein Using the Protein Chip

A sample containing labeled target proteins is added into each partition on the protein chip. The sample may contain fluorescent labels at 3′ end or 5′ end. From the detection of the label, the exact proteins in the sample can be identified.

While the invention has been particularly shown and described with the reference to the preferred embodiments thereof, it will be understood by those skilled in the art that various changes in form and details may be made without departing from the spirit and scope of the invention.

What is claimed is:

1. A preparation method for biochips, comprising:
(a) providing a substrate;
(b) applying a micro-injecting process to spray a hydrophobic material on the substrate for forming a hydrophobic region thereon, and a plurality of partitions being defined on the hydrophobic region; and
(c) immobilizing a probe on each partition by the micro-injecting process.

2. The preparation method as claimed in claim 1, wherein the hydrophobic material is selected from a group consisting of Teflon, polyimide, fluoro-compound, and silicon compound.

3. The preparation method as claimed in claim 1, wherein the micro-injecting process is performed by a micro-injector to spray vertically, horizontally, unidirectionally or bidirectioanlly.

4. The preparation method as claimed in claim 3, wherein the micro-injector is selected from a group consisting of a thermal bubble micro-injector and a piezo micro-injector.

5. The preparation method as claimed in claim 1, wherein the substrate is a hydrophobic substrate and is selected from a group consisting of glass, silica, quartz, mica, ceramics, and metals.

6. The preparation method as claimed in claim 5, further comprising a step (d), after the step (b), for forming a hydrophilic functional group on each partition.

7. The preparation method as claimed in claim 6, wherein the hydrophilic functional group is selected from a group consisting of —NH₂, —COOH, —SH, epoxide, aldehyde, and streptavidin.

8. The preparation method as claimed in claim 1, wherein the substrate is a hydrophilic substrate selected from a group consisting of polystyrene, polyester, polycarbonate, polyvinylchloride, polyethylene, polypropylene, polysulfone, polyurethane, and polymethylmethacrylate (PMMA).

9. The preparation method as claimed in claim 8, further comprising:
a step (e), after the step (a), hydrophobically treating the substrate; and
a step (f), after the step (b), hydrophilically treating each partition to form a hydrophilic functional group thereto.

10. The preparation method as claimed in claim 9, wherein the hydrophilic functional group is selected from a group consisting of —NH₂, —COOH, —SH, epoxide, aldehyde, and streptavidin.

11. The preparation method as claimed in claim 1, wherein the partitions are selected from a group consisting of square, circular, and geometric figures.

12. The preparation method as claimed in claim 1, wherein the probe is selected from a group consisting of DNA, RNA, nucleotides, oligonucleotides, protein, antibodies, and peptides.

13. The preparation method as claimed in claim 1, wherein the probe is immobilized to each partition by a binding process.

14. The preparation method as claimed in claim 13, wherein the binding process is selected from a group consisting of adsorption, covalent binding, encapsulation, cross-linking, and entrapment.

15. The preparation method as claimed in claim 1, wherein the micro injecting process is performed by a thermal micro-injector, and the micro-injector comprises:
a chamber for storing a fluid;
a micro injecting process pore disposed on the chamber for ejecting the fluid;
a first heater and a second heater arranged on two sides of the micro injecting process pore respectively;
when the chamber is full of the fluid, the first heater produces a first bubble and the second heater produces a second bubble, and the two bubbles spray out a drop of the fluid.

16. The preparation method as claimed in claim 15, wherein the first and the second heaters are triggered by one signal.

17. The preparation method as claimed in claim 15, wherein the first bubble acts as a valve to limit an ejection of the fluid in the chamber.

18. A biochip, comprising:
a substrate,
a plurality of hydrophobic regions formed on the substrate by micro-injecting a hydrophobic material on the substrate;
a plurality of hydrophilic partitions separated by the hydrophobic regions disposed on the substrate; and
a probe immobilized on each partition by a micro-injecting process.

19. The biochip as claimed in claim 18, wherein the substrate is a hydrophobic substrate selected from a group consisting of glass, silicon, quartz, mica, ceramics, and metals.

20. The biochip as claimed in claim 19, wherein the surface of the hydrophobic substrate contains a hydrophilic functional group after a hydrophilic treating.

21. The biochip as claimed in claim 20, wherein the hydrophilic functional group is selected from a group consisting of —NH₂, —COOH, —SH, epoxide, aldehyde, and streptavidin.

22. The biochip as claimed in claim 21, wherein the substrate is a hydrophilic substrate selected from a group consisting of polystyrene, polyester, polycarbonate, polyvinylchloride, polyethylene, polypropylene, polysulfone, polyurethane, and polymethylmethacrylate (PMMA).

23. The biochip as claimed in claim 20, wherein the substrate becomes hydrophobically because of a hydrophobic treatment performed on the substrate before the plurality of the partitions are formed.

24. The biochip as claimed in claim 23, wherein a hydrophilic treatment is performed on the partitions to add a hydrophilic functional group thereto after the partitions are formed.

25. The biochip as claimed in claim 24, wherein the hydrophilic functional group is selected from a group consisting of —NH₂, —COOH, —SH, epoxide, aldehyde, and streptavidin.

26. The biochip as claimed in claim 18, wherein the hydrophobic material is selected from a group consisting of Teflon, polyimide, compounds containing fluorides and silicides.

27. The biochip as claimed in claim 18, wherein the probe is selected from a group consisting of DNA, RNA, nucleotides, oligonucleotides, protein, antibodies, and peptides.

28. The biochip as claimed in claim 18, wherein the probe is immobilized on the partition by a process selected from a group consisting of adsorption, covalent binding, encapsulation, cross-linking, and entrapment.
